(19) United States
(12) Patent Application Publication
(10) Pub. No.: US 2001/0039439 A1
(43) Pub. Date: Nov. 8, 2001

(54) Compliant heat exchange splint and control unit

(76) Inventors: William Elkins, San Jose, CA (US); Kenneth L. Blanchard, Milpitas, CA (US)

Correspondence Address: ROSENLAW.COM, Suite 301, 702 Marshall Street, Redwood City, CA, 94063, US

(21) Appl. No.: 09/901,963

(22) Filed: Jul. 10, 2001

Related U.S. Application Data
(63) Continuation of application No. 09/173,637, filed on Oct. 16, 1998.

(51) Int. Cl. A61F 7/00

(52) U.S. Cl. 607/104; 607/114

(57) ABSTRACT

A heat exchange system using pressurized air and a flowing liquid having a controlled temperature for cooling or heating a portion of a human body. The system includes a compliant heat exchange splint and a dual function control unit. The splint includes a liquid bladder containing flowing liquid and an air bladder containing pressurized air for compressing the liquid bladder into close conformal contact with a body part of a person for cooling or heating the body part. The liquid and air bladders are defined by a common peripheral seal. The flowing liquid and the pressurized air enter and exit the splint directly into the liquid and air bladders through planar ports. The control unit includes a liquid circuit for providing the flowing fluid at a controlled temperature and an air circuit for providing the pressurized air. The control unit housing includes a coolant tub and a pivoting cover assembly that uses snaps for latching in closed positions for operation and in an open position for accessing the elements of the liquid and air circuits. The snaps in the closed position use mating protrusions and cavities, and in the open position a tongue and groove.

### BACKGROUND OF THE INVENTION

1. Field of the Invention

The invention relates generally to a heat exchange system having a compliant heat exchange splint and a control unit. More particularly the heat exchange splint includes a fluid bladder having a flowing fluid as a heat exchange medium and an air bladder having pressurized air for compressing the fluid bladder into close conformal contact with a person for cooling or heating a part of his body. The control unit includes a tub and a pivotal latching cover assembly for pumping the flowing fluid at a controlled temperature and the pressurized air.

2. Description of the Prior Art

Compliant heat exchange panels are used for cooling a portion of a human body for physical therapy, pre-game day conditioning, minor injury care, post orthoscopic surgery recovery, and as a replacement for general air-conditioning. Such panels use a heat absorbing medium having a lower temperature than the body for transferring heat from the body to the heat absorbing medium. The panel may be passive where the medium is stationary within the panel or active where the medium, typically a liquid, flows through the panel. In a passive panel the heat exchange medium is commonly frozen gel, ice packs, ice water, or even frozen peas. Passive heat exchange panels have the advantage of being very low in cost. However, such panels have several limitations. Ice water at 32° Fahrenheit or an ice pack at an even lower temperature is too cold for optimum use and can cause damage to human tissue. Further, the heat exchange medium must be changed or re-cooled when its temperature rises. Because the thermal mass of the heat absorbing medium in a passive panel is limited by the size of the panel, the operating time of the system is limited.

An active heat exchange system is more expensive than a passive system because an external control unit is required to pump and re-cool the liquid. However, an active heat exchange system is preferable for many applications because it can operate continuously over a long period of time while maintaining a controllable constant temperature. By warming the liquid in the external control unit, an active heat exchange system may also be used with the heat exchange panel for heating.

In order to achieve the best results in a heat exchange panel, the panel must have close conformal contact when it is wrapped about the body. This requirement is easier to meet when the panel is thin and flexible, however, some method is still required for holding the panel closely against the body. Belts and straps with adjustable lengths can be used for fastening the panel in place. However, this manner of fastening and holding the panel does not by itself ensure that the panel conforms to the nooks and crannies and complex curves of a portion of a human body. Several attempts have been made to overcome this problem with systems using air pressure and even cyclic air pressure for compressing the wrapped panel against the body. However, such existing systems are inconvenient to use and expensive.

It is sometimes desirable to use a heat exchange panel as a splint by wrapping the panel about a body part such as an arm or a leg. In one experiment by the present inventor about 1991 and 1992, a panel was used as a splint having both a liquid bladder for containing the flowing liquid and an air bladder for containing pressurized or cyclically pressurized air. The splint was constructed of three layers where a first layer intended to be adjacent to the body was sealed to a second layer for forming the edges of the liquid bladder and a third layer intended to be the outside of the splint was sealed to the first layer for forming the edges of the air bladder. The air bladder received the pressurized air through an air port at a right angle to the splint. The liquid bladder received and issued the flowing liquid through two fluid ports at a right angle to the splint having tubes in the air bladder for passing the flowing liquid. A control unit pumped the cooled liquid through the liquid bladder while pressurizing the air to the air bladder. The control unit included several boxes for batteries, pumps, a flow meter, a temperature controller, and a heat exchanger for passing the flowing liquid past a frozen canister. One of the improvements of this system over passive systems was that the operating time of the system was determined by the thermal mass of the heat exchange medium in the canister. Because the canister could easily be larger than a compliant heat exchange panel, this experimental system could be operated for a longer period of time than a passive system.

This experimental system had some success, however, the splint and the control unit had several limitations. One limitation was that the splint was relatively expensive due to the method of making the ports and seals. Another limitation was that the splint was potentially unreliable due to the right angle of the ports and the tubes in the air bladder for passing the flowing liquid. Still another limitation was that the splint did not make optimum thermal contact with the body due to buckling of the liquid bladder when the air bladder was pressurized. Further, the response time for the control unit for controlling the temperature of the liquid was limited due to the placement of the temperature controller before a reservoir. Another issue was that air pressure cycling depended upon air outflow in the splint thereby limiting the universality of the control unit. Another issue was that the control unit was expensive to manufacture and service due to the way in which the liquid and air circuit elements of the control unit were housed and assembled into the control unit. This system was not reproduced and/or commercialized.

### SUMMARY OF THE INVENTION

It is therefore an object of the present invention to provide an inexpensive and reliable compliant heat exchange splint that conforms to complex shapes for making good thermal contact.

Another object of the present invention is to provide an inexpensive and operationally convenient dual function control unit for pumping and controlling a temperature of a liquid, and controlling and cycling air pressure.

Briefly, in a preferred embodiment, a heat exchange system of the present invention includes a compliant heat exchange splint and a control unit. The heat exchange splint includes a liquid bladder and an air bladder. The liquid bladder is defined by a first layer, a second layer, and a peripheral seal. A dot matrix pattern of attachments between the first and second layers holds the liquid bladder in a thin shape for good compliance when wrapped about a body part such as an arm or a leg. The air bladder is defined by the other side of the second layer, a third layer, and the same peripheral seal. Liquid entry and exit ports and an air port are planar with the first, second, and third layers of the splint. The peripheral seal seals around the air and liquid ports and seals the first, second, and third layers together at the same time at a peripheral border that is common to both bladders. In operation, the splint is wrapped about the body part and fastened in place with a pressure-sensitive fastener on the inside of an extended section beyond the peripheral seal of the first layer that attaches to a mating fastening surface covering most of the outside of the third layer. The third layer including the mating fastening surface is elastic so that the air bladder can expand with the pressured air without causing buckling in the liquid bladder. A flowing liquid enters the liquid bladder through the liquid entry port, passes through the liquid bladder and exits through the liquid exit port, and air is pressurized into the air bladder through the air port.

The control unit includes a liquid circuit and an air circuit. The liquid circuit includes a reservoir for storing a liquid, a liquid pump for pumping the liquid from the reservoir through the liquid bladder and back to the reservoir, and a temperature controller disposed between the liquid pump and the liquid bladder for controlling the temperature of the liquid. The temperature controller controls the temperature of the liquid by controlling the portion of the liquid that passes through a heat exchange coil immersed in a fluid coolant and the portion of the liquid that bypasses the coil. The air circuit includes an air pump for pumping pressurized air into the air bladder, a pressure controller for turning the air pump on and off for cycling air pressure, and a bleed orifice disposed between the air pump and the air bladder for bleeding the pressurized air.

The control unit is housed with a coolant tub and a pivoting cover assembly. The cover assembly has a closed position for covering the top of the tub and an open position for enabling an operator to access the inside of the tub. The coil and the coolant are disposed in the tub. The reservoir, the liquid pump, the temperature controller, the air pump, the pressure controller, and the bleed orifice are housed in the cover assembly. A tongue on a raised panel of the cover assembly mates with a groove on a handle on the back outer side of the tub for latching the cover assembly in the open position in order to prevent the cover assembly or raised panel from inadvertently closing and hitting the operator. The cover assembly latches in the closed position with a cover snap using a cavity on a front edge of the cover assembly and a mating tub snap using a protrusion on the front inner side of the tub. In the closed position, a protrusion on a rear edge of the cover assembly pushes the cover assembly forward against the back inner side of the tub in order to urge the snaps together and improving their holding strength. The cover assembly includes a tray, a lid above the tray, and the raised panel above the lid that are pivotally engaged to each other and to the tub for accessing the elements of the liquid and air circuits. The tray, the lid, and the raised panel latch together with snaps using protrusions and mating cavities.

An advantage of the present invention of a compliant heat exchange splint of is that it is low in cost and reliable by having air and liquid bladders that are sealed with a peripheral seal that is common to both bladders; and having planar air and liquid ports for passing air and liquid through the peripheral seal directly into the air and liquid bladders, respectively.

Another advantage of the compliant heat exchange splint of the present invention is that it has an elastic outer layer in order to substantially avoid buckling of the liquid bladder when the air bladder is inflated, thereby making a better thermal contact for exchanging heat with a body part and ensuring a more uniform liquid flow throughout the splint.

An advantage of the present invention is that it provides a flowing liquid at a controlled temperature and a cycling pressurized air from a single control unit.

Another advantage of the present invention of the control unit is that it is low in cost and convenient for service and operation by using a housing having a pivoting cover assembly that closes for operation and opens for service.

Another advantage of the control unit of the present invention is that it is inexpensive and convenient by using automatic snaps for latching into a closed position for operation and an open position for service.

Another advantage of the control unit of the present invention is that it has a fast temperature response time by disposing a liquid temperature controller between a liquid pump and a heat exchange splint.

Another advantage of the control unit of the present invention is that it includes a bleed orifice for cycling air pressure without depending upon an air outflow in a heat exchange splint.

These and other objects and advantages of the present invention will no doubt become obvious to those of ordinary skill in the art after having read the following detailed description of the preferred embodiments which are illustrated in the various figures.

### BRIEF DESCRIPTION OF THE DRAWINGS

FIGS. 1a and 1b are plan and cross-sectional drawings, respectively, of a compliant heat exchange splint of the present invention; and

FIG. 2 is a block diagram of a control unit of the present invention for operating the splint of FIG. 1;

FIGS. 3 and 4 are a perspective views of the control unit of FIG. 2 showing a cover assembly in closed and open positions, respectively;

FIG. 5 is a cross-sectional view of the control unit of FIG. 2 showing a tray, a lid, and a raised panel of the cover assembly in open positions; and

FIG. 6 is an exploded perspective view of the control unit of FIG. 2.

### DETAILED DESCRIPTION OF THE PREFERRED EMBODIMENTS

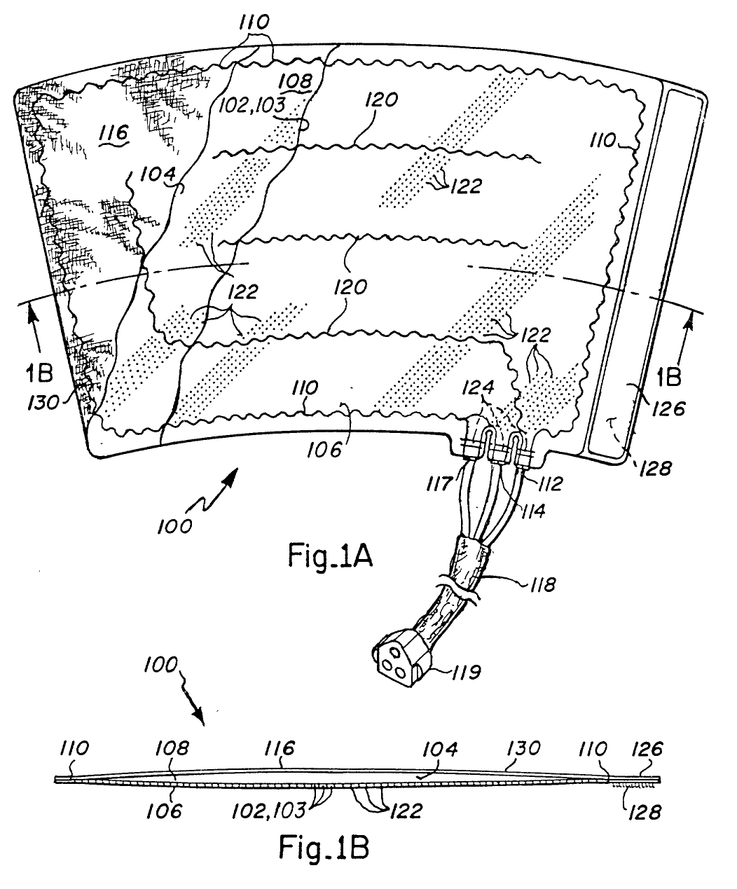

FIGS. 1a and 1b are a plan and cross-sectional drawings, respectively, of a compliant heat exchange splint of the present invention referred to by the general reference number 100. The splint 100 includes a liquid bladder 102 for containing a flow of a liquid 103 at a controlled temperature and an air bladder 104 for containing pressurized air. Natural atmospheric air is preferred, however, other pure or mixed gases can be used. In use, the splint 100 is wrapped so that the liquid bladder 102 is in contact with a body or portion of a body. The liquid 103 that flows through the liquid bladder 102 picks up or gives out heat from or to the body depending upon whether the temperature of the liquid 103 is lower or greater, respectively, than the skin temperature of the body. The pressurized in the air bladder 104 compresses the liquid bladder 102 against the body part to better conform to its shape. In a preferred application, the body part is a person's leg or arm. However, the body may be some other animate or inanimate object. Typically, with a human body, the splint 100 is used for cooling with a temperature for the liquid 103 in a range of 45° to 50° Fahrenheit. Preferably, the liquid 103 is a 20% propylene glycol solution in distilled water with a small amount of wetting agent for breaking surface tension and an anti-fungicide such as iodine, however, other fluids may be used.

The liquid bladder 102 is defined by a first layer 106, a second layer 108, and a peripheral seal 110. A liquid entry port 112 and a liquid exit port 114 through the peripheral seal 110 enable the liquid 103 to be pumped through the liquid bladder 102. It is not critical which of the liquid ports 112 and 114 is the liquid entry port 112 and which is the liquid exit port 114. The air bladder 104 is defined by the other side of the second layer 108, a third layer 116 and the peripheral seal 110. The same peripheral seal 110 is used for both the liquid bladder 102 and the air bladder 104. An air pressure port 117 connects through the peripheral seal 110 to enable the air bladder 104 to be pressured. The liquid ports 112 and 114 and the air port 117 connect into respective hoses in a cable 118 to respective openings in a liquid/air connector 119.

The first layer 106 and the second layer 108 are sealed together at fences 120 and at attachment dots 122 of a dot matrix. The first, second, and third layers 106, 108, and 116 are then sealed together with the peripheral seal 110. The peripheral seal 110 continues across tubes for the liquid entry and exit ports 112 and 114 and the air pressure port 117 with double ring seals 124 for extra reliability. The fences 120 and the attachment dots 122 enable the liquid bladder 102 to retain a thin planar shape in use while the liquid 103 is being pumped to flow. The air bladder 104 is allowed to expand when it is pressurized.

The attachment dots 122 are arranged in a pattern of squares so that lines from any one of the attachment dots 122 to its nearest four neighbors are separated by 90° angles, thereby minimizing strain on the first and second layers 106 and 108 and providing the thinnest and most compliant splint 100 for a given number of attachment dots 122. The lines connecting nearest neighbors of attachment dots 122 cross at approximately 45° to the flow of the liquid 103, thereby reducing the widths of clear channels for the flow of the liquid 103 and increasing mixing of the liquid 103 as it flows in order to maintain a more even temperature for the liquid 103 throughout the area of the liquid bladder 102. The fences 120 channel the liquid 103 to ensure that the liquid 103 passes through the entire area of the liquid bladder 102. Both the peripheral seal 110 and the fences 120 are rippled in order to minimize compressive stresses and buckling when the bladders 102 and 104 are inflated and to improve the compliance of the splint 100 when it is wrapped about a complex shape. The ripples are smooth in order to minimize slow areas of trapped liquid 103 that would result in warm areas within the liquid bladder 102.

The first layer 106, the second layer 108, and the third layer 116 may be constructed of a laminate having a fabric and one or more coatings of a material that can be heat welded for making a seal. Preferably, the coatings are a thermal plastic, such as an ether-based polyurethane. In the first layer 106 the coatings are on the side facing into the liquid bladder 102. In the third layer 116 the coatings are on the side facing into the air bladder 104. The second layer 108 is coated on both sides. In a preferred embodiment, the coatings of the first and third layers 106 and 116 have three plies. The first ply applied to the fabric has a relatively low density, the second ply has a relatively high density, and the third ply has a relatively low density. The second layer 108 may be constructed in a manner that is similar using two or three plies on each side. Nylon having a woven two-hundred denier rating is suitable for the fabric of the first and second layers 106 and 108. The fabric for the third layer 116 is elastic in order to minimize buckling of the first and second layers 106 and 108 when the air bladder 104 is expanded by the pressurized air and the splint 100 is wrapped about the body. Preferably, the fabric for the third layer 116 is a stretchable nylon or polyester knit having an outer surface material similar to VELCRO™ loops. Preferable laminated fabrics are available from Highland Industries of Framingham, Mass., as a model L004 for the first layer 106, a model B005 for the second layer 108, and a model S053 for the third layer 116; the sealing process for the fences 120, the attachment dots 122, and the peripheral border 110 is a Radio Frequency (RF) heat sealing process available from Ocean Vendors of Byron, Calif.; and plates for the RF heat sealing process are made of Magnesium material using a photo engraving process available from Custom Photo Engraving of Redwood City, Calif.

The first layer 106 includes an extended section 126 extending beyond the peripheral seal 110. The extended section 126 is stiffened by a high density polyethylene sheet pocketed for assembly. The inward side (the side that faces toward the body when the splint 100 is used as a wrap) of the extended section 126 includes a first pressure-sensitive fastener 128, preferably a VELCRO™ hook strip. The outward surface of the third layer 116 has an elastic second pressure-sensitive fastener 130 similar to VELCRO™ loops for mating to the first pressure-sensitive fastener 128. In operation, the splint 100 is wrapped about a human body part, such as an arm, a leg, or the like, with the ports 112, 114, and 117 at the distal end of the body part and then held in place by the pressure-sensitive fasteners 128 and 130. The placement of the fastener 128 on the fastener 130 enables the diameter of the splint 100 to be adjusted to fit the size of the body part. The liquid 103 is then pumped into the liquid entry port 112, through the liquid bladder 102, and out of the liquid exit port 114. Preferably, the liquid 103 is pumped at about 10-15 psig. Air is pumped through the air pressure port 117 into the air bladder 104 in order to urge the splint 100 and especially the liquid 103 in the liquid bladder 102 into close conformal proximity with the selected body part. Preferably, the air pressure is cycled up to an excess pressure (pressure above atmospheric pressure) of about eighty to one-hundred ten millimeters of Mercury for about one or two minutes and then down to an excess pressure of about ten to thirty millimeters with a total cycle time of about three to five minutes in order to move edema toward the core of the body and to ensure blood flow beneath the splint 100. Although, typically a controlled temperature of the liquid 103 of about 45° to 50° degrees Fahrenheit is desirable when using the splint 100 for injury recovery and pre-game preparation, the splint 100 may be used with colder or warmer temperatures or for heating. The close conformal proximity and the lower temperature of the liquid 103 causes heat to be transferred between the body part and the liquid 103.

Even though the operation of the splint 100 has been described in terms of an active heat exchanger having a flowing liquid 103 as a heat exchange medium, the splint 100 can be used as a passive heat exchanger where the heat exchange medium is periodically exchanged or the splint 100 is periodically removed and re-cooled as required for maintaining a desired temperature. Typically, the heat exchange medium in the passive splint 100 should be a flexible material in a phase change such as a gel, a mix having ice particles in liquid, or small solid ice particles.

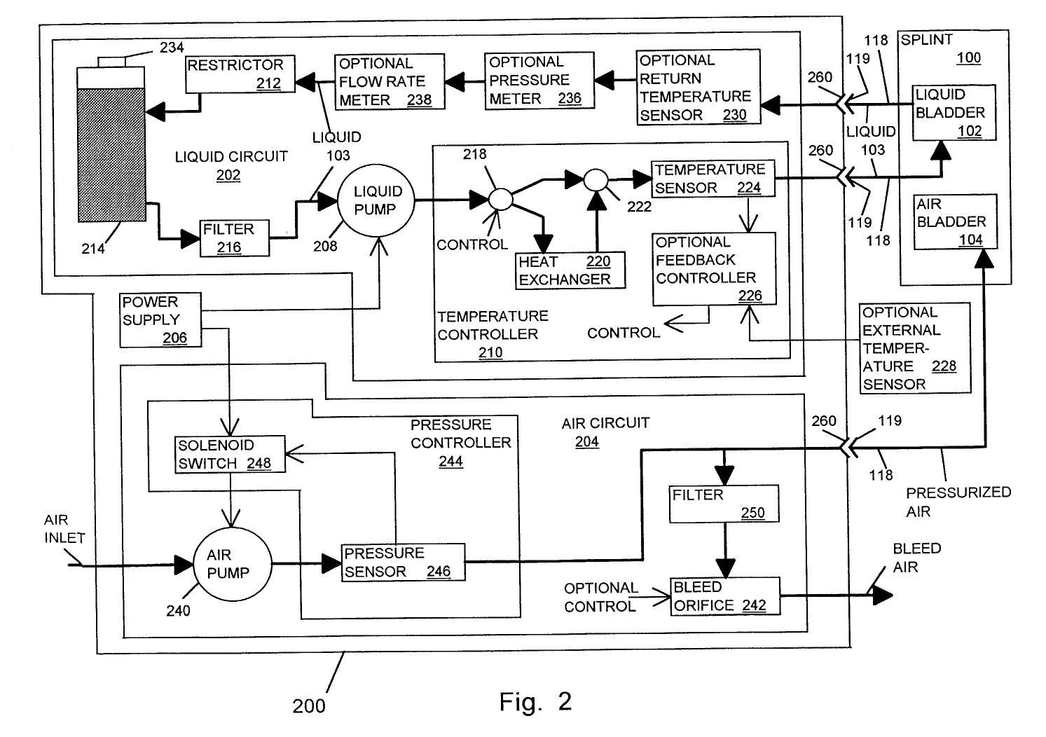

FIG. 2 is block diagram of a control unit of the present invention referred to by the general reference number 200. The control unit 200 includes a liquid circuit 202 for pumping the liquid 103 at a controlled temperature through the liquid bladder 102 of the splint 100, an air circuit 204 for cycling pressure in the air bladder 104 of the splint 100, and a power supply 206. Preferred implementations for the types and construction details of the various structural elements used in the liquid circuit 202, the air circuit 204, and the power supply 206 are described below. However, it should be obvious to those having ordinary skill in the art that alternative types and construction details are suitable for the structural elements in the present invention.

The liquid circuit 202 includes a liquid pump 208 for pumping the liquid 103, a temperature controller 210 for controlling the temperature of the liquid 103, a restrictor 212 for regulating the pressure of the liquid 103, a sealed flexible reservoir 214 for storing excess liquid 103, and a filter 216 for cleaning the liquid 103. Preferably, the liquid 103 is a 20% propylene glycol solution in distilled water with a small amount of wetting agent for breaking surface tension and an anti-fungicide such as iodine, however, other fluids may be used. The liquid pump 208 includes an electrical motor powered from the power supply 206 for pumping the liquid 103 to the temperature controller 210. The temperature controller 210 includes a mixing valve 218 including a Y junction for splitting the liquid 103 from the liquid pump 208 into two tubes and a cam having a controllable angular rotation for alternatively closing or opening the two tubes with peristaltic action, thereby selectively passing more or less portions of the liquid 103 into two paths. A first path passes through a heat exchanger 220 having an ice water bath for cooling the liquid 103 to a temperature approaching as low as 32° Fahrenheit. From the heat exchanger 220 the liquid 103 in the first path flows to a Y-junction 222. The liquid 103 in the second path flows directly to the Y-junction 222. The temperature of the liquid 103 at the outflow of the Y-junction 222 is determined by the portions and temperatures of the liquid 103 flowing through each of the paths.

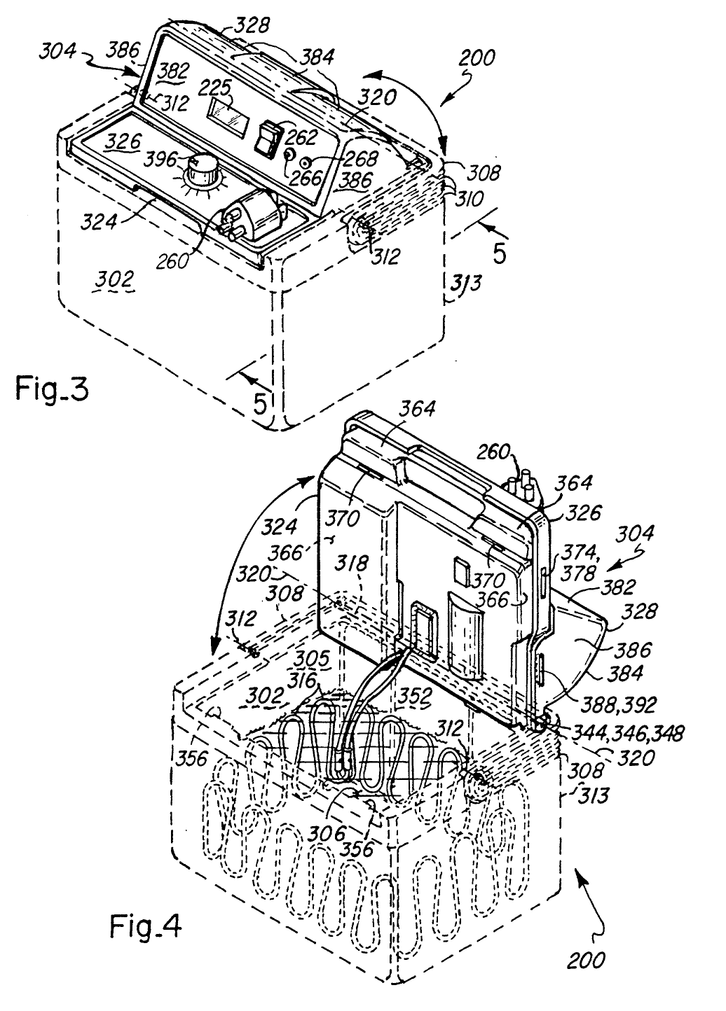

After the Y-junction 222 the liquid 103 flows to a temperature sensor 224. In a preferred embodiment, the temperature sensor 224 includes a temperature readout 225 (FIGS. 3, 5, and 6) and the mixing valve 218 operates under manual control by a person. The person provides the temperature feedback control by reading the temperature readout 225 (FIGS. 3, 5, and 6) and manually controlling the portion of the liquid 103 flowing through the first and second paths in order to obtain a desired temperature. However, several alternative embodiments are optional. An optional automatic feedback controller 226 may be used for adjusting the mixing valve 218 for a selected temperature based upon temperature information received from the temperature sensor 224, from an optional external temperature sensor 228, from an optional return temperature sensor 230, or from a combination of temperature sensors. The external temperature sensor 228 may be used to provide biofeedback for the temperature, for example a rectal temperature, of a body being cooled by the splint 100.

The liquid 103 from the temperature sensor 224 flows through the liquid bladder 102 to the restrictor 212. The restrictor 212 uses a spring-loaded valve to maintain a positive pressure of the liquid 103 in the liquid bladder 102 to a range of roughly one-half to one atmosphere. The restrictor 212 passes the liquid 103 to the reservoir 214. The reservoir 214 opens with a reservoir cap 234 threaded onto the reservoir 214 to enable an operator to add liquid 103 to the liquid circuit 202, stores excess liquid 103, and allows trapped air to separate. From the reservoir 212 the liquid 103 passes to the filter 216. The filter 216 uses a fine pore material for protecting the liquid pump 208 from particles. The liquid pump 208 receives the liquid 103 from the filter 216, thereby completing a loop for the flowing liquid 103. A pressure meter 236 with a readout and a flow rate meter 238 with a readout and are optional.

The air circuit 204 includes an air pump 240 for providing pressurized air to the air bladder 104, a bleed orifice 242 for bleeding the pressurized air, and a pressure controller 244 for repetitively cycling the pressurized air between a high pressure level and a low pressure level. The air pump 240 receives outside air from an air inlet and pressurizes a volume of about one or two liters in about one-half to two minutes. From the air pump 240 the pressurized air passes to the pressure controller 244. The pressure controller 244 includes a pressure sensor 246 and a solenoid switch 248. The pressure sensor 246 issues a first electrical sensor signal to the solenoid switch 248 when air pressure is or falls below the low pressure level and issues a second electrical sensor signal to the solenoid switch 248 when the air pressure rises to or above the high pressure level. The solenoid switch 248 responds to the first sensor signal by providing an electrical power connection for passing electrical power from the power supply 206 to the air pump 240 and responds to the second sensor signal by breaking the power connection to the air pump 240. The air pump 240 includes an electrical motor for using the electrical power for pressurizing the inlet air. Alternatively, the bleed orifice 242 may be controlled by the pressure sensor 246 while the air pump 240 operates continuously. Such controllable bleed orifice 242 may be constructed using a needle valve.

The pressurized air from the pressure sensor 246 passes by a filter 250 and on to the air bladder 104 in the splint 100. A portion of the pressurized air passes through the filter 250 to the bleed orifice 242. The bleed orifice 242 includes an exit hole for slowly bleeding the pressurized air from the high pressure level to the low pressure level over a time period of two to five minutes. In a preferred embodiment the high pressure level is about eighty to one-hundred ten millimeters of Mercury, the low pressure level is about ten to thirty millimeters of Mercury, and the exit hole of the orifice 242 is about six to ten thousandths inch diameter. Optionally, the pressure sensor 246 includes a pressure readout and a control input for reading and controlling the high or the low pressure level. The pressure sensor 246 may use a spring for sensing air pressure and may have control of the air pressure that is sensed pre-compressing or expanding the spring.

Liquid 103 pumped from the liquid circuit 202 to the liquid bladder 102, liquid 103 returned from the liquid bladder 102 to the liquid circuit 202, and air pumped and returned between the air circuit 204 and the air bladder 104 pass through respective connections in a connector 260. The connections in the connector 260 mate with respective connections in the connector 119 (FIG. 1a) and through respective hoses in the cable 118 to the splint 100.

The power supply 206 includes a master on/off switch 262 (FIGS. 3, 5, and 6), a battery pack 264 (FIG. 3), a circuit breaker switch 266 (FIGS. 3, 5, and 6), and a low voltage AC input connection 268 ((FIGS. 3, 5, and 6). The low voltage AC input connection 268 (FIGS. 3, 5, and 6) is used with a transformer from an AC power line. Alternatively, the power supply 206 may be powered from AC line or through an automobile adapter.

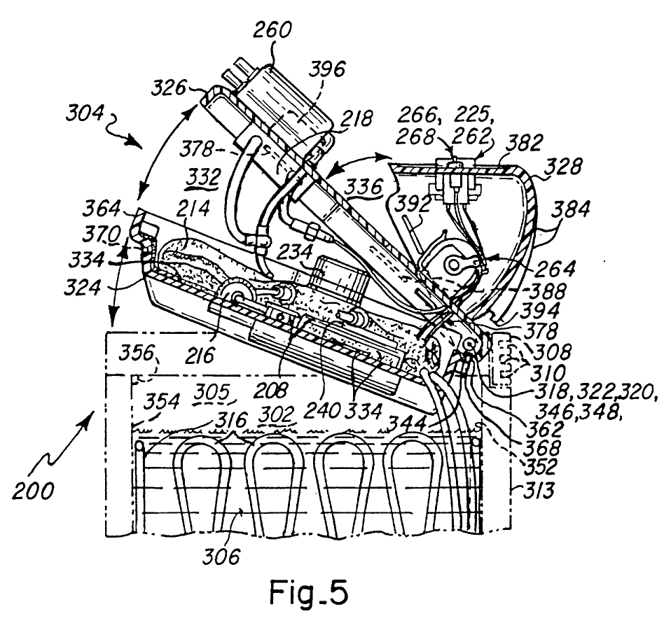

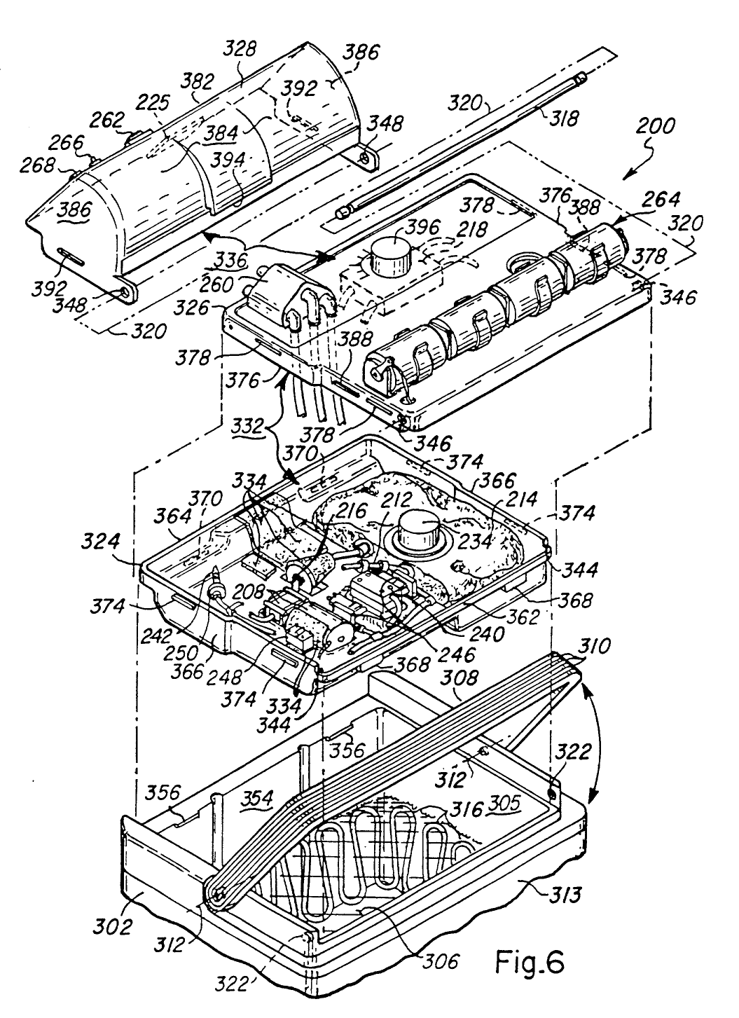

FIGS. 3 and 4 are perspective views of the control unit 200 for controlling an external heat exchanger such as the heat exchange splint 100 (FIGS. 1a-b). FIGS. 5 and 6 are cross-sectional and exploded views, respectively, of the control unit 200. The control unit 200 includes a coolant tub 302 having thermal insulation and a pivoting cover assembly 304. The connector 260 includes connections that mate with connections in connector 119 (FIG. 1a) for issuing the pressurized air and issuing and receiving the flowing fluid 103 (FIG. 2) to and from the external heat exchanger.

The cover assembly 304 pivots between a closed cover position as shown in the FIG. 3 for covering an open top side 305 of the tub 302 and an open cover position as shown in the FIG. 4 for exposing and allowing access to the inside of the tub 302. Preferably, the tub 302 is made with plastic. Such tub 302 is commercially available as model Legend 12 from Igloo Products Corp. of Katy, Tex. A coolant 306, preferably a mix of ice and liquid water, is loaded into the inside of the tub 302. Water in the ice-liquid phase change is preferred as the coolant 306 because it is inexpensive and has a temperature (about 32° F.) that is less than the preferred temperature of 45° F. to 50° F. for the flowing liquid 103 (FIG. 2), however, other materials can be used depending upon the requirements of the application. The tub 302 includes a handle 308 having grooves 310. The handle 308 pivots about a pivot axis 312 for stowage against a back outer side 313 of the tub 302 as shown in FIGS. 3, 4, and 5 or for carrying as shown in FIG. 6. A drain plug (not shown) may be built into a side of the tub 302 for draining the tub 302. A suitable drain plug is available from Rubbermade Gott, Inc. of Winfield, Tex.

The heat exchanger 220 (FIG. 2) includes a heat exchange coil 316 for conducting the flowing liquid 103 (FIG. 2) from the mixing valve 218 to the Y-connection 222 (FIG. 2). The coil 316 is disposed in the coolant 306 for exchanging heat between the flowing liquid 103 (FIG. 2) and the coolant 306. Preferably the coil 316 is constructed of stainless steel, however, other heat conducting materials can be used.

The cover assembly 304 is pivotally engaged to the tub 302 with a pivot rod 318 along a cover pivot axis 320 for pivoting between the closed cover position and the open cover position. In the open cover position the inside of the tub 302 is accessible in order to change the coolant 306. The ends of the pivot rod 318 are retained in the tub 302 for rotation in blind pivot holes 322. The cover assembly 304 includes a tray 324, a lid 326 above the tray 324, and a raised panel 328 above the lid 326. Preferably, the housings are vacuum formed or injection molded plastic. A first chamber 332 between the tray 324 and the lid 326 houses the liquid pump 208, the reservoir 214, the reservoir cap 234, the restrictor 212 (inside tube entering the reservoir 214), the filter 216, the mixing valve 218, the air pump 240, the bleed orifice 242, the pressure sensor 246, the solenoid switch 248, the filter 250, and several foam pieces 334. The foam pieces 334 are used for holding the components in the first chamber 332 in place without the use of fastenings. A second chamber 336 between the lid 326 and the raised panel 328 houses the battery pack 264. The battery 264 may include separate batteries as shown or several batteries in a combining package. The pivot rod 318 includes annular slots for bearing on steps 344 in the tray 324 and the pivot rod 318 passes through holes 346 in the lid 326 and holes 348 in the raised panel 328, thereby enabling the raised panel 328, the lid 326, and the tray 324 to pivot together as a unit with respect to the tub 302 as shown in the FIGS. 3 and 4 and to pivot separately with respect to each other as shown in the FIG. 5 to allow service access to the first and second chambers 332 and 336. Alternately, in a preferred embodiment, U-shaped slots may be used in place of the steps 344 for bearing the pivot rod 318.

The tub 302 includes a back inner side 352 that is adjacent and parallel to the pivot axis 320 and a forward inner side 354 opposite and parallel to the back inner side 352. The forward inner side 354 includes a tub snap 356. Preferably, the tub snap 356 uses rearward projecting and horizontally elongated protrusions molded into the tub 302. The tray 324 includes a tray rear edge 362 adjacent to the back inner side 352 of the tub 302, a tray forward edge 364 opposite and parallel to the tray rear edge 362, and tray side edges 366 between and perpendicular to the tray rear edge 362 and the tray forward edge 364. The tray rear edge 362 includes tray rear edge protrusions 368 and the tray forward edge 364 includes a cover snap 370. Preferably, the cover snap 370 uses horizontally elongated cavities either molded or machined into the tray 324. When the cover assembly 304 pivots into the closed cover position and/or the tray 324 pivots into position for closing the open top side 305 of the tub 302, the tub snap 356 mates and latches with the cover snap 370 and the tray rear edge protrusions 368 press against the back inner side 352 of the tub 302, thereby pushing the tray 324 forward in order to ensure that the latching action between the tub snap 356 and the cover snap 370 is secure.

The tray side edges 366 include a tray snap 374. Preferably, the tray snap 374 uses horizontally elongated outward projecting protrusions molded into the tray 324. The lid 326 includes lid side edges 376 adjacent, parallel, and outside the tray side edges 366. The lid side edges 376 include a first lid snap 378. Preferably, the first lid snap 378 uses horizontally elongated cavities molded or machined into the lid 326. The tray snap 374 mates with the first lid snap 378 for latching the tray 324 and the lid 326 together in a closed position. The raised panel 328 includes an angled front panel 382, a curved rear panel 384, and side panels 386 that is adjacent, parallel, and outside the lid side edges 376. The lid side edges 376 further include a second lid snap 388 preferably using horizontally elongated outward projecting protrusions molded in the lid 326. The side panels 386 include a panel snap 392, preferably using horizontally elongated cavities. The lid snap 388 mates and latches with the panel snap 392 for latching the lid 326 and the panel 328 together in a closed position. The curved rear panel 384 includes a horizontally elongated overhanging tongue 394. When the cover assembly 304 pivots into an open cover position or the raised panel 328 pivots into an open position for exposing and allowing access to the second chamber 336, the tongue 394 and one of the grooves 310 in the handle 308 on the back outer side 313 of the tub 302 act as a snap latch combination for holding is the cover assembly 304 and/or the raised panel 328 in an open position. The second chamber 336 includes the battery pack 264 that must be periodically replaced for service. The handle 308 is effectively a part of the back outer side 313 of the tub 302 by a press fit of the handle 308 to the back outer side 313 of the tub 302 and an offset between the handle pivot axis 312 and the cover pivot axis 320.

The shapes and materials of the tub 302, the handle 308, the tray 324, the lid 326, and the raised panel 328 enable the mating snaps described above to spring together and spring apart for automatically latching and unlatching as the cover assembly 304, the tray 324, the lid 326, and the raised panel 328 are opened and closed. The snaps 356, 370, 374, 378, 388, and 392 use mating protrusions and cavities in a preferred embodiment because such protrusions and cavities are simply and inexpensively molded or machined in plastic materials. Similarly, the tongue 394 and grooves 310 are preferred as snap latches because they are simply and inexpensively molded in plastic. Further, the snap 356 and the grooves 310 are preferred because of their availability in a standard commercial version of the tub 302 described above. However, it should be understood that the cavities used for snaps 370, 378, and 392 and protrusions used for snaps 356, 374, and 388 described in the preferred embodiment above may be reversed. Further, the cavities used for snaps 370, 378, and 392 may be implemented as through holes or recesses.

The front panel 382 includes the temperature readout 225, the master on/off switch 262 having two positions and a light emitting diode indicating when the electrical power of the power supply 206 (FIG. 2) to the control unit 200 is turned on or off, the circuit breaker switch 266, and the low voltage AC input connection 268. The lid 324 includes an operable temperature control knob 396 for controlling the temperature of the flowing liquid 103 (FIG. 2) by controlling the mixing valve 218.

Although the present invention has been described in terms of the presently preferred embodiments, it is to be understood that such disclosure is not to be interpreted as limiting. Various alterations and modifications will no doubt become apparent to those skilled in the art after having read the above disclosure. Accordingly, it is intended that the appended claims be interpreted as covering all alterations and modifications as fall within the true spirit and scope of the invention.

What is claimed is:

1. A compliant heat exchanger, comprising:
a first bladder having a heat exchange medium having a temperature for exchanging said heat energy with a body, the first bladder defined by a first layer, a second layer, and a peripheral seal;
a second bladder having pressurized air for compressing the first bladder against said body, said second bladder defined by said second layer, a third layer, and said peripheral seal; and
said peripheral seal for sealing said first layer, said second layer, and said third layer at a peripheral border common to said first, second, and third layer.

2. The heat exchanger of claim 1, wherein:
said heat exchange medium is at least one of a gel, a mix of liquid and solid particles, solid particles, or a fluid.

3. The heat exchanger of claim 1, wherein:
said heat exchange medium is a flowing fluid.

4. The heat exchanger of claim 1, further comprising:
at least two fluid ports through said peripheral seal and planar with said first and second layers for passing said heat exchange medium through said first bladder.

5. The heat exchanger of claim 1, further comprising:
at least one air port through said peripheral seal and planar with said second and third layers for passing said pressurized air into said second bladder.

6. The heat exchanger of claim 1, wherein:
said pressurized air is repetitively cycled between at least two pressure levels.

7. The heat exchanger of claim 1, wherein:
said third layer is elastic for stretching when said second bladder is pressurized for reducing buckling in said first and second layers.

8. The heat exchanger of claim 1, further comprising:
an extended section of said first layer extending beyond said peripheral seal; and
a pressure-sensitive fastener on said first layer for adjustably fastening to the outside of said third layer for holding the heat exchanger against said body when the heat exchanger is wrapped about said body.

9. The heat exchanger of claim 1 wherein:
said first layer includes a sealable coating on the side facing the first bladder;
said second layer includes a sealable coating on the side facing said first bladder and a sealable coating on the side facing said second bladder;
said third layer includes a sealable coating on the side facing the second bladder; and
said peripheral seal is for sealing said first, second, and third layers together at said common peripheral border when sealable coatings are heated at said common peripheral border.

10. A control unit for controlling a heat exchanger having a fluid bladder and an air bladder, comprising:
a fluid circuit including a fluid pump for pumping a fluid through said fluid bladder and a temperature controller disposed between said fluid pump and said fluid bladder for controlling a temperature of said fluid; and
an air circuit including an air pump for pressurizing air to said air bladder.

11. The control unit of claim 10, wherein:
the air circuit further includes cycling means for repetitively cycling said pressurized air between at least two pressure levels.

12. The control unit of claim 11, wherein:
said cycling means includes a pressure controller for turning on said air pump when said pressurized air is less than a first pressure and turning off said air pump when said pressurized air is greater than a second pressure, and a bleed orifice disposed between said air pump and said air bladder for bleeding said pressurized air, whereby said pressurized air decreases from said first pressure to said second pressure when said air pump is turned off.

13. A method in a compliant heat exchanger for exchanging heat energy with a body, comprising steps of:
sealing a first layer, a second layer, and a third layer with a peripheral seal at a peripheral border common to said first, second, and third layer;
providing a heat exchange medium in a first bladder defined by said first layer, said second layer, and said peripheral seal; said heat exchange medium having a temperature for exchanging said heat energy with said body; and
pressurizing air in a second bladder for compressing said first bladder against said body; said second bladder defined by said second layer, said third layer, and said peripheral seal.

14. The method of claim 13, wherein:
said heat exchange medium is at least one of a gel, a mix of liquid and solid particles, solid particles, or a fluid.

15. The method of claim 13, wherein:
said heat exchange medium is a flowing fluid.

16. The method of claim 13, wherein:
the step of providing said heat exchange medium includes steps of: passing said heat exchange medium through said peripheral seal into said first bladder through a first fluid port planar with said first and second layers; and passing said heat exchange medium through said peripheral seal out of said first bladder through a second fluid port planar with said first and second layers.

17. The method of claim 13, wherein:
the step of pressurizing air includes passing pressurized air through said peripheral seal into said second bladder through an air port planar with said second and third layers.

18. The method of claim 13, wherein:
the step of pressurizing air includes repetitively cycling pressurized air between at least two pressure levels.

19. The method of claim 13, wherein:
the step of pressurizing air includes stretching said third layer for reducing buckling in said first and second layers.

20. The method of claim 13, further comprising steps of:
wrapping said heat exchanger about a part of said body with said first bladder adjacent to said body part; and
adjustably fastening said first layer to the outside of said third layer with a pressure-sensitive fastener for holding said heat exchanger wrapped about said body part.

21. The method of claim 13, further comprising steps of:
constructing said first layer with a sealable material on the side facing into said first bladder;
constructing said second layer with a sealable material on the side facing into said first bladder and a sealable material on the side facing into said second bladder;
constructing said third layer with a sealable material on the side facing into said second bladder; and
sealing said first, second, and third layers together at said common peripheral border by heating said sealable materials.

22. A method of controlling a heat exchanger having a fluid bladder and an air bladder, comprising steps of:
pumping a fluid through a temperature controller to said fluid bladder;
controlling a temperature of said fluid with said temperature controller; and
pressurizing air to said air bladder.

23. The method of claim 22, wherein:
the step of pressurizing air includes a step of repetitively cycling said pressurized air between at least two pressure levels.

24. The method of claim 23, wherein:
the step of cycling said pressurized air includes turning on an air pump when said pressurized air is less than a first pressure; turning off said air pump when said pressurized air is greater than a second pressure; and bleeding said pressurized air through an orifice disposed between said air pump and said second bladder.

25. A control unit for controlling an external heat exchanger, comprising:
a tub having an open top side;
a cover assembly including a fluid pump for pumping a fluid through said external heat exchanger, the cover assembly pivotally engaged to the tub about a cover pivot axis and having a closed cover position for covering said open top side and an open cover position for uncovering said open top side for allowing access to the inside of the tub.

26. The control unit of claim 25, wherein:
the cover assembly further includes a reservoir coupled for supplying said fluid to said fluid pump, a temperature controller for receiving said fluid from said fluid pump and controlling a temperature of said fluid, and a temperature sensor for measuring a temperature of said fluid.

27. The control unit of claim 25, further comprising:
a coolant disposed in the tub;
a coil disposed in the coolant; and wherein:
said fluid pump is further for pumping said fluid through said coil for transferring heat energy between said external heat exchanger and said coolant.

28. The control unit of claim 25, wherein:
the cover assembly further includes an air pump for pumping pressurized air to said external heat exchanger.

29. The control unit of claim 28, wherein:
the cover assembly further includes a bleed orifice for bleeding said pressurized air.

30. The control unit of claim 25, wherein:
the tub includes a back outer side having a groove parallel to said cover pivot axis; and
the cover assembly includes a raised panel having a tongue parallel to said cover pivot axis for latching to said groove for holding said cover assembly in said open cover position.

31. The control unit of claim 30, further including:
a handle having a stowage position disposed on said outer back side of said tub, said handle including said groove.

32. The control unit of claim 25, wherein:
the tub includes a back inner side adjacent to said cover pivot axis and a forward inner side opposite said back inner side, said forward inner side having a tub snap; and
the cover assembly includes a rear edge adjacent to said back inner side and a forward edge adjacent to said forward inner side in said closed cover position, said forward edge having a cover snap for latching with said tub snap in said cover closed position, and further comprising:
a protrusion on one of (i) said back inner side of the tub or (ii) said rear edge of the cover assembly for pressing said cover snap against said tub snap for latching the cover assembly in said closed cover position.

33. The control unit of claim 32, wherein:
said tub snap is one of (i) a protrusion and (ii) a cavity; and
said cover snap is the other of (i) a protrusion and (ii) a cavity.

34. The control unit of claim 25, wherein:
the cover assembly includes a tray and a lid pivotally engaged to each other for opening or closing a first chamber between said tray and said lid; and
said fluid pump is disposed in said first chamber.

35. The control unit of claim 34, wherein:
said tray includes a tray edge having a tray snap; and
said lid includes a lid edge having a first lid snap for latching to said tray snap when said first chamber is closed.

36. The control unit of claim 35, wherein:
said tray snap is one of (i) a protrusion and (ii) a cavity; and
said first lid snap is the other of (i) a protrusion and (ii) a cavity.

37. The control unit of claim 34, wherein:
the cover assembly further includes a raised panel pivotally engaged to said lid for opening or closing a second chamber between said lid and said raised panel;
said lid includes a lid edge having a second lid snap;
said raised panel includes a panel edge having a panel snap for latching to said second lid snap when said second chamber is closed.

38. A heat exchange system, comprising:
a heat exchange splint including a fluid bladder having a flowing fluid having a temperature for exchanging heat energy with a body; and
a control unit including a tub having an open top side and a cover assembly having a fluid pump disposed in said cover assembly for pumping a fluid through said fluid bladder, the cover assembly pivotally engaged to the tub about a cover pivot axis and having a closed cover position for covering said top side and an open cover position for uncovering said open top side for allowing access to the inside of the tub.

39. The system of claim 38, wherein:
the heat exchange splint further includes an air bladder; and
said cover assembly further includes an air pump disposed in said cover assembly for pumping pressurized air to said air bladder for compressing said fluid bladder against said body.

40. The system of claim 39, wherein:
said fluid bladder is defined by a first layer, a second layer, and a peripheral seal;
said air bladder is defined by the other side of said second layer, a third layer, and said peripheral seal
said peripheral seal is for sealing said first layer, said second layer, and said third layer at a peripheral border common to said first, second, and third layer.

41. The system of claim 38, further comprising:
a coolant disposed in said tub;
a coil disposed in the coolant; and wherein:
said fluid pump is further for pumping said fluid through said coil for transferring heat energy between said fluid and said coolant, whereby said heat energy from said body is transferred to said coolant.

42. The system of claim 38, wherein:
said cover assembly includes a tray and a lid pivotally engaged to each other for opening or closing a first chamber between said tray and said lid, said first chamber for housing said fluid pump.

43. The system of claim 42, wherein:
said tray includes a tray edge having a tray snap; and
said lid includes a lid edge having a lid snap for latching to said tray snap when said first chamber is closed.

44. The system of claim 38, wherein:
said tub includes an outer back side having a groove parallel to said cover pivot axis; and
said cover assembly includes a raised panel having a tongue parallel to said cover pivot axis for latching to said groove for holding said cover assembly in said open cover position.

45. The system of claim 44, wherein:
said cover assembly further includes a lid;
said lid and said raised panel are pivotally engaged to each other for opening or closing a second chamber between said lid and said raised panel, said second chamber for housing a battery for powering said fluid pump.
